(19) United States
(12) Patent Application Publication
(10) Pub. No.: US 2024/0409611 A1
(43) Pub. Date: Dec. 12, 2024

(54) VEGFR-FC FUSION PROTEIN FORMULATIONS

(71) Applicant: Amgen Inc., Thousand Oaks, CA (US)

(72) Inventors: Yael WEXLER-COHEN, Thousand Oaks, CA (US); Robert Matthew FESINMEYER, Newbury Park, CA (US); Monica Michelle GOSS, Newbury Park, CA (US); Sekhar KANAPURAM, Thousand Oaks, CA (US); Rahul Rajan KAUSHIK, Newbury Park, CA (US); Sai Chakradhar PADALA, Thousand Oaks, CA (US)

(73) Assignee: Amgen Inc., Thousand Oaks, CA (US)

(21) Appl. No.: 18/817,735

(22) Filed: Aug. 28, 2024

Related U.S. Application Data
(63) Continuation of application No. 17/932,255, filed on Sep. 14, 2022, now abandoned.
Application No. 17/932,255 is a continuation of application No. 16/647,902, filed on Mar. 17, 2020, now abandoned.
(60) Provisional application No. 62/618,910, filed on Jan. 18, 2018.
(60) Provisional application No. 62/559,987, filed on Sep. 18, 2017.

(51) Int. Cl. C07K 14/71; A61K 9/00

(52) U.S. Cl. CPC C07K 14/71; A61K 9/0019; C07K 2319/31

(57) ABSTRACT

VEGFR-Fc fusion protein formulations and methods of making using such formulations are provided herein. In one embodiment, the formulation is an ophthalmic formulation, such as for intravitreal administration. In some embodiments, the VEGFR-Fc fusion protein is aflibercept.

### RELATED APPLICATIONS

This application is a continuation of U.S. application Ser. No. 17/932,255 filed on Sep. 14, 2022, which is a continuation of U.S. application Ser. No. 16/647,902 filed on Sep. 17, 2020, which is a U.S. national stage filing under 35 U.S.C. § 371 of PCT Application Number PCT/US2018/051311, filed on Sep. 17, 2018, which claims the benefit of U.S. Provisional Application No. 62/559,987, filed on Sep. 18, 2017, and U.S. Provisional Application No. 62/618,910, filed on Jan. 18, 2018, each of which is hereby incorporated by reference in its entirety.

### SEQUENCE LISTING

The present application is being filed along with a Sequence Listing in electronic format. The Sequence Listing is provided as a file entitled A-2197-US06-CNT_SQL, created Aug. 28, 2024, which is 4 kb in size. The information in the electronic format of the Sequence Listing is incorporated herein by reference in its entirety.

### FIELD OF THE INVENTION

The instant disclosure relates to VEGFR-Fc fusion protein formulations and methods for making and using such formulations.

### BACKGROUND

Vascular endothelial growth factor (VEGF), also referred to as VEGF-A, is a signaling protein that promotes the growth of new blood vessels and binds to VEGFR-1 and VEGFR-2. VEGF has been shown to be upregulated in many tumors and has a role in angiogenesis. VEGF has also been shown to have a role in intraocular neovascularization, such as choroidal neovascularization (CNV), which is a significant aspect of wet age-related macular degeneration (AMD).

VEGF inhibitors, such as anti-VEGF antibodies and fragments and decoy receptors or chimeric receptors, have been developed as therapeutics for the treatment of various conditions, such as cancer and ocular disorders. For example, an anti-VEGF antibody and an anti-VEGF Fab are both commercially available as bevacizumab and ranibizumab, respectively. Also, commercially available is aflibercept, a VEGFR-Fc fusion protein or “VEGF-trap.”

Aflibercept is a fusion protein composed of an IgG1 Fc domain fused to the Ig domain 2 of VEGFR-1 and Ig domain 3 of VEGFR-2. Aflibercept is marketed as Eylea® (Regeneron, Tarrytown, NY) for the treatment of various ocular conditions, including wet type AMD, and is formulated for intravitreal administration. The fusion protein is also marketed as Zaltrap® (ziv-aflibercept) (Regeneron, Tarrytown, NY) for the treatment of certain types of cancer and is formulated for intravenous administration.

Ophthalmic formulations, and in particular intravitreal administration, can have additional safety concerns as compared to other administration routes, and thus, have more specific requirements. For example, impact to a subject due to inflammation or other adverse reactions can be severe, and thus, more specific requirements may be required. For example, a formulation for intravitreal administration may require a narrower range of permissible osmolarity. A formulation for intravitreal administration may require a lower threshold of permissible particulation, e.g., USP <789> versus USP <788>. It is also advantageous to have a formulation that provides increased stability.

The present disclosure provides formulations that meets the need for new VEGF formulations that are stable, have less aggregation, and/or have related advantages.

### SUMMARY

Provided herein are VEGFR-Fc fusion protein formulations and methods for making and using such formulations. In one embodiment, the formulation comprises a fusion protein comprising a domain of a vascular endothelial growth factor (VEGF) receptor and an Fc domain, a buffer, a stabilizer, and optionally, a surfactant. In some embodiments, the formulation has a pH below 6.0, below 5.9, below 5.8, below 5.7, below 5.6, below 5.5, below 5.4, below 5.3, below 5.2 or below 5.1. In some embodiments, the pH is between 5.0 and 6.0, between 5.0 and 5.9, between 5.0 and 5.8, between 5.0 and 5.7, between 5.0 and 5.6, between 5.0 and 5.5, between 5.1 and 6.0, between 5.1 and 5.9, between 5.1 and 5.8, between 5.1 and 5.7, between 5.1 and 5.6, between 5.1 and 5.5, between 5.2 and 6.0, between 5.2 and 5.9, between 5.2 and 5.8, between 5.2 and 5.7, between 5.2 and 5.6, between 5.2 and 5.5, between 5.3 and 6.0, between 5.3 and 5.9, between 5.3 and 5.8, between 5.3 and 5.7, between 5.3 and 5.6, or between 5.3 and 5.5. In some embodiments, the pH is about 6.0, about 5.9, about 5.8, about 5.7, about 5.6, about 5.5, about 5.4, about 5.3, about 5.2 or about 5.1. In some embodiments, the pH is 6.0±0.3, 5.9±0.3, 5.8±0.3, 5.7±0.3, 5.6±0.3, 5.5±0.3, 5.4±0.3, 5.3±0.3, 5.2±0.3 or 5.1±0.3.

In one embodiment, the formulation comprises a fusion protein comprising a domain of a vascular endothelial growth factor (VEGF) receptor and an Fc domain, a histidine buffer, a stabilizer, and optionally, a surfactant; and has a pH below 5.7. In one embodiment, the formulation comprises a fusion protein comprising a domain of a vascular endothelial growth factor (VEGF) receptor and an Fc domain, a phosphate buffer, a stabilizer, and optionally, a surfactant; and has a pH below 6.0. In another embodiment, the formulation comprises a fusion protein comprising a domain of a vascular endothelial growth factor (VEGF) receptor and an Fc domain, an acetate buffer, a stabilizer, and optionally, a surfactant; and has a pH below 5.6.

In one embodiment, the formulation comprises: aflibercept, a histidine or phosphate buffer, a stabilizer, and optionally, a surfactant; and has a pH below 5.7. In another embodiment, the formulation comprises: aflibercept, an acetate buffer, a stabilizer, and optionally, a surfactant, and has a pH below 5.6. In some embodiments, the concentration of aflibercept is about 40 mg/ml.

### BRIEF DESCRIPTION OF THE DRAWINGS

FIG. 1 shows the correlation between aflibercept aggregation level and pH for the formulations described in Table 3 after 4 weeks at 25° C.

### DETAILED DESCRIPTION

The instant disclosure provides VEGFR-Fc fusion protein formulations and methods for making and using such formulations. In one embodiment, the formulation comprises a fusion protein comprising a domain of a vascular endothelial growth factor (VEGF) receptor and an Fc domain (e.g., aflibercept), a buffer, stabilizer, and optionally, a surfactant. In some embodiments, the formulation has a pH below 6.0, below 5.9, below 5.8, below 5.7, below 5.6, below 5.5, below 5.4, below 5.3, or below 5.2. In some embodiments, the pH is between 5.0 and 6.0, between 5.0 and 5.9, between 5.0 and 5.8, between 5.0 and 5.7, between 5.0 and 5.6, between 5.0 and 5.5, between 5.1 and 6.0, between 5.1 and 5.9, between 5.1 and 5.8, between 5.1 and 5.7, between 5.1 and 5.6, between 5.1 and 5.5, between 5.2 and 6.0, between 5.2 and 5.9, between 5.2 and 5.8, between 5.2 and 5.7, between 5.2 and 5.6, between 5.2 and 5.5, between 5.3 and 6.0, between 5.3 and 5.9, between 5.3 and 5.8, between 5.3 and 5.7, between 5.3 and 5.6, or between 5.3 and 5.5. In some embodiments, the pH is about 6.0, about 5.9, about 5.8, about 5.7, about 5.6, about 5.5, about 5.4, about 5.3, about 5.2 or about 5.1. In some embodiments, the pH is 6.0±0.3, 5.9±0.3, 5.8±0.3, 5.7±0.3, 5.6±0.3, 5.5±0.3, 5.4±0.3, 5.3±0.3, 5.2±0.3 or 5.1±0.3.

In one embodiment, the formulation comprises between 1 and 100 mg/ml of a fusion protein comprising a domain of a vascular endothelial growth factor (VEGF) receptor and an Fc domain, a buffer, stabilizer, and optionally, a surfactant. In some embodiments, the formulation comprises between 1 and 50 mg/ml of the fusion protein. In one embodiment, the formulation comprises between 10 and 50 mg/ml of the fusion protein. In one embodiment, the formulation comprises about 40 mg/ml of the fusion protein.

In some embodiments, the formulation comprises a fusion protein comprising a domain of a vascular endothelial growth factor receptor (VEGFR) and an Fc domain. In one embodiment, the fusion protein comprises a domain of VEGFR1, a domain of VEGFR2, or a combination thereof. In some embodiments, the fusion protein comprises a domain of VEGFR1 and a domain of VEGFR2. In one embodiment, the fusion protein comprises Ig domain 2 of VEGFR1 and Ig domain 3 of VEGFR2. In one embodiment, the fusion protein comprises Ig domain 2 of VEGFR1, Ig domain 3 of VEGFR2, and an Fc domain of IgG1. In one embodiment, the fusion protein is a VEGF Trap. In another embodiment, the fusion protein is aflibercept. In one embodiment, the fusion protein comprises an amino acid sequence of SEQ ID NO: 1. In another embodiment, the fusion protein comprises an amino acid sequence of SEQ ID NO: 2. In one embodiment, the formulation comprises about 40 mg/ml of aflibercept. In one embodiment, the formulation comprises about 40 mg/ml of a fusion protein comprising a protein having an amino acid sequence of SEQ ID NO: 1 or SEQ ID NO: 2. In one embodiment, the formulation comprises about 40 mg/ml of a fusion protein comprising a protein having an amino acid sequence of SEQ ID NO: 1 and a fusion protein comprising a protein having an amino acid sequence of SEQ ID NO: 2.

SEQ ID NO: 1
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 2
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGK
(SEQ ID NO: 2)

The fusion protein can be produced by any suitable method known in the art, such as described in U.S. Pat. No. 7,070,959.

In one embodiment, the formulation comprises a fusion protein comprising a domain of a vascular endothelial growth factor (VEGF) receptor and an Fc domain (e.g., aflibercept), a buffer, stabilizer, and a surfactant, wherein the buffer is a phosphate buffer. In one embodiment, the phosphate buffer is a potassium phosphate buffer. In another embodiment, the phosphate buffer is a sodium phosphate buffer. In one embodiment, the concentration of the phosphate buffer is between 1 mM to 50 mM, between 1 mM to 40 mM, between 1 mM to 30 mM, between 1 mM to 20 mM, between 1 mM to 10 mM, or between 1 mM to 5 mM. In one embodiment, the concentration of the phosphate buffer is about 1 mM, about 5 mM, about 10 mM, about 20 mM, about 30 mM, about 40 mM, or about 50 mM.

In one embodiment, the formulation comprises a fusion protein comprising a domain of a vascular endothelial growth factor (VEGF) receptor and an Fc domain (e.g., aflibercept), a buffer, stabilizer, and optionally, a surfactant, wherein the buffer is a histidine buffer. The histidine buffer can be produced from the non-salt form of histidine or the salt form of histidine. In one embodiment, the histidine buffer comprises a histidine salt, such as histidine-HCl. In another embodiment, the histidine buffer comprises histidine acetate. In one embodiment, the concentration of the histidine buffer is between 1 mM to 50 mM, between 1 mM to 40 mM, between 1 mM to 30 mM, between 1 mM to 20 mM, between 1 mM to 10 mM, or between 1 mM to 5 mM. In one embodiment, the concentration of the histidine buffer is about 1 mM, about 5 mM, about 10 mM, about 20 mM, about 30 mM, about 40 mM, or about 50 mM. In some embodiments, the pH is between 5.0 and 6.0, between 5.0 and 5.9, between 5.0 and 5.8, between 5.0 and 5.7, between 5.0 and 5.6, between 5.0 and 5.5, between 5.1 and 6.0, between 5.1 and 5.9, between 5.1 and 5.8, between 5.1 and 5.7, between 5.1 and 5.6, between 5.1 and 5.5, between 5.2 and 6.0, between 5.2 and 5.9, between 5.2 and 5.8, between 5.2 and 5.7, between 5.2 and 5.6, between 5.2 and 5.5, between 5.3 and 6.0, between 5.3 and 5.9, between 5.3 and 5.8, between 5.3 and 5.7, between 5.3 and 5.6, or between 5.3 and 5.5. In another embodiment, the pH is between 5.7 and 5.9. In another embodiment, the pH is between 5.0 and 5.7. In another embodiment, the pH is between 5.3 and 5.7. In one embodiment, the formulation has a pH below 5.9. In one embodiment, the pH is about 5.9, about 5.8, about 5.7, or about 5.6, about 5.5, about 5.4, about 5.3, about 5.2 or about 5.1. In one embodiment, the pH is about 5.9. In one embodiment, the pH is about 5.8. In one embodiment, the pH is about 5.7. In another embodiment, the pH is about 5.5. In yet another embodiment, the pH is about 5.2. In some embodiments, the pH is 5.9±0.3, 5.8±0.3, 5.7±0.3, 5.6±0.3, 5.5±0.3, 5.4±0.3, 5.3±0.3, 5.2±0.3 or 5.1±0.3.

In one embodiment, the formulation comprises a fusion protein comprising a domain of a vascular endothelial growth factor (VEGF) receptor and an Fc domain (e.g., aflibercept), a buffer, stabilizer, and optionally, a surfactant, wherein the buffer is an acetate buffer. The acetate buffer can be produced from the non-salt form of acetate or the salt form of acetate. In one embodiment, the acetate buffer can comprise an acetate salt or acetic acid, such as glacial acetic acid. In another embodiment, the acetate buffer comprises sodium acetate. In one embodiment, the concentration of the acetate buffer is between 1 mM to 50 mM, between 1 mM to 40 mM, between 1 mM to 30 mM, between 1 mM to 20 mM, between 1 mM to 10 mM, or between 1 mM to 5 mM. In one embodiment, the concentration of the acetate buffer is about 1 mM, about 2.5 mM, about 5 mM, about 10 mM, about 20 mM, about 30 mM, about 40 mM, or about 50 mM. In some embodiments, the pH is between 5.0 and 6.0, between 5.0 and 5.9, between 5.0 and 5.8, between 5.0 and 5.7, between 5.0 and 5.6, between 5.0 and 5.5, between 5.1 and 6.0, between 5.1 and 5.9, between 5.1 and 5.8, between 5.1 and 5.7, between 5.1 and 5.6, between 5.1 and 5.5, between 5.2 and 6.0, between 5.2 and 5.9, between 5.2 and 5.8, between 5.2 and 5.7, between 5.2 and 5.6, between 5.2 and 5.5, between 5.3 and 6.0, between 5.3 and 5.9, between 5.3 and 5.8, between 5.3 and 5.7, between 5.3 and 5.6, or between 5.3 and 5.5. In one embodiment, the pH is between 5.0 and 5.8. In another embodiment, the pH is between 5.0 and 5.7. In another embodiment, the pH is between 5.3 and 5.7. In one embodiment, the formulation has a pH below 5.8. In one embodiment, the pH is about 5.8, about 5.7, about 5.6, about 5.5, about 5.4, about 5.3, about 5.2 or about 5.1. In one embodiment, the pH is about 5.5. In one embodiment, the pH is about 5.2. In some embodiments, the pH is 5.8±0.3, 5.7±0.3, 5.6±0.3, 5.5±0.3, 5.4±0.3, 5.3±0.3, 5.2±0.3 or 5.1±0.3.

In one embodiment, the formulation comprises a fusion protein comprising a domain of a vascular endothelial growth factor (VEGF) receptor and an Fc domain (e.g., aflibercept), a buffer (e.g., a phosphate, histidine, or acetate buffer as described above), a stabilizer, and optionally, a surfactant, wherein the stabilizer is an amino acid or sugar. In one embodiment, the stabilizer is an amino acid. In one embodiment, the amino acid is proline. In another embodiment, the amino acid is glycine. In yet another embodiment, the amino acid is arginine. In one embodiment, the stabilizer is a sugar. The sugar can be sucrose, sorbitol, glycerol, trehalose, mannitol, dextrose, glucose or any combination thereof. In one embodiment, the stabilizer is sucrose. In another embodiment, the stabilizer is trehalose, such as α,α-trehalose dihydrate. In yet another embodiment, the formulation comprises two different sugars, such as sucrose and trehalose. In another embodiment, the stabilizer is cyclodextrin.

The concentration of the stabilizer can be between 1 mM to 300 mM, between 10 mM to 300 mM, between 100 mM to 300 mM, between 200 mM to 300 mM, and between 200 mM and 280 mM. In one embodiment, the concentration of the stabilizer is about 200 mM, such as about 200 mM proline. In another embodiment, the concentration of the stabilizer is about 280 mM, such as about 280 mM glycine. In yet another embodiment, the concentration of the stabilizer is about 165 mM, such as about 165 mM arginine.

In yet other embodiments, the formulation comprises between 0 and 20% (w/v) of the stabilizer. In some embodiments, the formulation comprises between 0 and 10% (w/v) or between 5 and 10% (w/v). In some embodiments, the formulation comprises about 5%, about 6%, about 7%, about 8%, about 9%, or about 10% (w/v) of a stabilizer, such as a sugar. In one embodiment, the formulation comprises about 5%, about 6%, about 7%, about 8%, about 9%, or about 10% (w/v) of sucrose. In yet another embodiment, the formulation comprises about 5%, about 6%, about 7%, about 8%, about 9%, or about 10% (w/v) of trehalose. In yet another embodiment, the formulation comprises about 5%, about 6%, about 7%, about 8%, about 9%, or about 10% (w/v) of sucrose and trehalose, such as about 1% (w/v) sucrose and about 7% (w/v) trehalose, about 2% (w/v) sucrose and about 6% (w/v) trehalose, about 3% (w/v) sucrose and about 5% (w/v) trehalose, about 4% (w/v) sucrose and about 4% (w/v) trehalose, about 5% (w/v) sucrose and about 4% (w/v) trehalose, about 5% (w/v) sucrose and about 3.5% (w/v) trehalose, about 5% (w/v) sucrose and about 3% (w/v) trehalose, about 6% (w/v) sucrose and about 2% (w/v) trehalose, about 7% (w/v) sucrose and about 1% (w/v) trehalose, or about 5% (w/v) sucrose and about 4% (w/v) trehalose.

In one embodiment, the formulation comprises a fusion protein comprising a domain of a vascular endothelial growth factor (VEGF) receptor and an Fc domain (e.g., aflibercept), a buffer (e.g., a phosphate, histidine, or acetate buffer as described above), a stabilizer (e.g., an amino acid or sugar, such as trehalose, sucrose, or a combination thereof), and a surfactant, wherein the surfactant is a polyoxyethylene glycol alkyl ether, a polyoxypropylene glycol alkyl ether, a glucoside alkyl ether, a polyoxyethylene glycol octylphenol ether, a polyoxyethylene glycol alkylphenol ether, a glycerol alkyl ester, a polyoxyethylene glycol sorbitan alkyl ester, a sorbitan alkyl ester, a cocamide MEA, a cocamide DEA, a dodecyldimethylamine oxide, a poloxamer, a polyethoxylated tallow amine (POEA), or a combination thereof. In one embodiment, the surfactant is a polysorbate. In one embodiment, the surfactant is polysorbate 20. In another embodiment, the surfactant is polysorbate 80. In yet another embodiment, the surfactant is a poloxamer, such as poloxamer 188. In one embodiment, the surfactant is Pluronic® F-68. In some embodiments, the formulation comprises between 0.001 to 0.5% (w/v) or between 0.01% to 0.1% (w/v) of a surfactant. In some embodiments, the formulation comprises about 0.01% (w/v) of a surfactant, such as polysorbate 80. In some embodiments, the formulation comprises about 0.01% (w/v) of a surfactant, such as polysorbate 20. In some embodiments, the formulation comprises about 0.005% (w/v) of a surfactant, such as polysorbate 80. In some embodiments, the formulation comprises about 0.03% (w/v) of a surfactant, such as polysorbate 20. In some embodiments, the formulation comprises about 0.1% (w/v) of a surfactant, such as Pluronic® F-68.

In on embodiment, the formulation comprises a fusion protein comprising a domain of a vascular endothelial growth factor (VEGF) receptor and an Fc domain (e.g., aflibercept), a histidine buffer, sucrose and/or trehalose, and a polysorbate. In one embodiment, the formulation comprises about 10 mM histidine buffer, about 8% (w/v) sucrose, and about 0.03% (w/v) polysorbate 20, at a pH of about 5.5. In another embodiment, the formulation comprises about 10 mM histidine buffer, about 4% (w/v) sucrose, about 4% trehalose, and about 0.01% (w/v) polysorbate 80, at a pH of about 5.8. In yet another embodiment, the formulation comprises about 10 mM histidine buffer, about 4% (w/v) sucrose, about 4% trehalose, and about 0.01% (w/v) polysorbate 80, at a pH of about 5.5. In one embodiment, the formulation comprises about 10 mM histidine buffer, about 4% (w/v) sucrose, about 4% trehalose, and about 0.1% (w/v) Pluronic® F68, at a pH of about 5.5. In another embodiment, the formulation comprises about 10 mM histidine buffer, about 9% trehalose, and about 0.01% (w/v) polysorbate 80, at a pH of about 5.8. In another embodiment, the formulation comprises about 10 mM histidine buffer, about 10% trehalose, and about 0.01% (w/v) polysorbate 20, at a pH of about 5.5. In some embodiments, the formulation comprises about 40 mg/ml of the fusion protein (e.g., aflibercept).

In one embodiment, the formulation comprises a fusion protein comprising a domain of a vascular endothelial growth factor (VEGF) receptor and an Fc domain (e.g., aflibercept), an acetate buffer, sucrose and/or trehalose, and a polysorbate. In one embodiment, the formulation comprises about 2.5 mM acetate buffer, about 5% (w/v) sucrose, about 3% (w/v) trehalose, and about 0.01% (w/v) polysorbate 80, at a pH of about 5.5. In one embodiment, the formulation comprises about 2.5 mM acetate buffer, about 5% (w/v) sucrose, about 4% (w/v) trehalose, and about 0.01% (w/v) polysorbate 80, at a pH of about 5.5. In one embodiment, the formulation comprises about 10 mM acetate buffer, about 5% (w/v) sucrose, about 3.5% (w/v) trehalose, and about 0.01% (w/v) polysorbate 80, at a pH of about 5.5. In one embodiment, the formulation comprises about 10 mM acetate buffer, about 5% (w/v) sucrose, about 3% (w/v) trehalose, and about 0.01% (w/v) polysorbate 80, at a pH of about 5.5. In one embodiment, the formulation comprises about 10 mM acetate buffer, about 5% (w/v) sucrose, about 4% (w/v) trehalose, and about 0.01% (w/v) polysorbate 80, at a pH of about 5.5. In one embodiment, the formulation comprises about 10 mM acetate buffer, about 9% (w/v) sucrose, and about 0.01% (w/v) polysorbate 80, at a pH of about 5.5. In some embodiments, the formulation comprises about 40 mg/ml of the fusion protein (e.g., aflibercept).

In some embodiments, the formulation comprises a fusion protein comprising a domain of a vascular endothelial growth factor (VEGF) receptor and an Fc domain (e.g., aflibercept), a buffer, stabilizer, and a tonicity agent, and optionally, a surfactant. The concentration of the tonicity agent can be between 1 mM to 250 mM, between 5 mM to 200 mM, between 40 mM to 200 mM, or between 40 mM to 140 mM. In one embodiment, the concentration of the tonicity agent is about 5 mM, about 10 mM, about 20 mM, about 30 mM, about 40 mM, about 50 mM, about 100 mM or about 140 mM. The tonicity agent can be a salt, such as a chloride salt. In one embodiment, the tonicity agent is sodium chloride. In another embodiment, the tonicity agent is potassium chloride.

In one embodiment, the formulation comprises an acetate buffer, sucrose, trehalose, a tonicity agent, and a polysorbate. In one embodiment, the formulation comprises about 2.5 mM acetate buffer, about 5% (w/v) sucrose, about 3% (w/v) trehalose, about 5 mM sodium chloride, and about 0.01% (w/v) polysorbate 80, at a pH of about 5.5. In one embodiment, the formulation comprises about 2.5 mM acetate buffer, about 5% (w/v) sucrose, about 4% (w/v) trehalose, about 5 mM sodium chloride, and about 0.01% (w/v) polysorbate 80, at a pH of about 5.5. In one embodiment, the formulation comprises about 10 mM acetate buffer, about 5% (w/v) sucrose, about 3.5% (w/v) trehalose, about 5 mM sodium chloride, and about 0.01% (w/v) polysorbate 80, at a pH of about 5.5. In one embodiment, the formulation comprises about 10 mM acetate buffer, about 5% (w/v) sucrose, about 4% (w/v) trehalose, about 5 mM sodium chloride, and about 0.01% (w/v) polysorbate 80, at a pH of about 5.5. In some embodiments, the formulation comprises about 40 mg/ml of the fusion protein (e.g., aflibercept).

In some embodiments, the formulation disclosed herein are used for intravitreal administration, such as for the treatment of an ocular condition such as wet type age related macular degeneration (AMD). In one embodiment, the formulation is capable to be used with a prefilled syringe. In one embodiment, the prefilled syringe is for intravitreal administration of the formulation.

In some embodiments, the formulation disclosed herein has a particulate count of less than 100 particles, less than 75 particles, less than 50 particles, less than 25 particles, less than 20 particles, less than 15 particles, less than 10 particles, or less than 5 particles, per one milliliter, for a particle size of ≥10 μm. In some embodiments, the formulation disclosed herein has a particulate count of less than 100 particles, less than 75 particles, less than 50 particles, less than 25 particles, less than 20 particles, less than 15 particles, less than 10 particles, or less than 5 particles, per one milliliter, for a particle size of ≥25 μm. In some embodiments, the formulation disclosed herein has a particulate count of less than 50 particles per one milliliter for particle size of ≥10 μm. In some embodiments, the formulation disclosed herein has a particulate count of less than 5 particles per one milliliter for particle size of ≥25 μm. In some embodiments, the formulation disclosed herein has a particulate count of no more than an average of 50 particles per one milliliter for particle size of ≥10 μm. In some embodiments, the formulation disclosed herein has a particulate count of no more than an average of 5 particles per one milliliter for particle size of ≥25 μm.

In some embodiments, a first formulation is determined to be more stable than a second formulation when the fusion protein of the first formulation retains more of its original characteristics or properties than the fusion protein of the second formulation after one or more process stresses and/or after storage for a given time period. Stability of a formulation can be determined by analyzing the properties or characteristics of the protein such as known in the art, for example, as described in U.S. Pat. Nos. 8,092,803 and 9,982,032, and PCT Publications WO2017129685 and WO2018094316.

In one embodiment, a first formulation is determined to be more stable than a second formulation when the first formulation has less aggregation than the second formulation after one or more process stresses or stress conditions, such as known in the art, e.g., as described in WO2017129685. In one embodiment, the stress condition is shaking. In another embodiment, the stress condition is one or more freeze/thaw cycles, such as one, two, three, four or five freeze/thaw cycles. In another embodiment, the stress condition is vibration, pressure, and/or drop-shock. In one embodiment, the stress condition is photoexposure. In one embodiment, the stress condition is mixing. In one embodiment, the formulation is subjected to any one or more of the stress conditions. The stress conditions can comprise shaking, one or more freeze/thaw cycle(s), filtration, mixing, photoexposure, vibration, pressure, drop-shock stress, and/or any combination thereof. In one embodiment, the stress process comprises shaking (e.g., at 300 rpm at 25° C. for seven days); three freeze/thaw cycles from 25° C. to −20° C. with a rate of 1° C./min, and after each cooling/heating step the temperature is kept constant for ten minutes. In another embodiment, the stress process comprises three freeze/thaw cycles between 25° C. to −30° C.; filtration through a 0.2 μm PVDF filter; optionally, mixing; holding at 2° C. to 8° C., photoexposure, and a full transportation simulation (e.g., with a 91.5 h sequence, which includes vibration, pressure and drop-shock stresses).

In another embodiment, a first formulation is determined to be more stable than a second formulation when the first formulation has less aggregation than the second formulation after storage for about 1 week, about two weeks, about 3 weeks, about 4 weeks, about 3 months, about 4 months, about 5 months, about 6 months, about 7 months, about 8 months, about 9 months, about 10 months, about 11 months, about 12 months, about 15 months, about 18 months, about 21 months, or about 24 months. Storage can be at a given temperature, e.g., about 40° C., about 25° C., about 5° C., or about −30° C.

In one embodiment, a formulation is more stable than a second formulation when the first formulation has less aggregation than the second formulation after one or more process stresses and storage for a given time period (e.g., about 1 week, about two weeks, about 3 weeks, about 4 weeks, about 3 months, about 4 months, about 5 months, about 6 months, about 7 months, about 8 months, about 9 months, about 10 months, about 11 months, about 15 months, about 18 months, about 21 months, or about 24 months at about 40° C., about 25° C., about 5° C., or about −30° C.)

The stability of a formulation can be performed by any method known in the art, such as described in U.S. Pat. Nos. 8,092,803 and 9,982,032, and PCT Publications WO2017129685 and WO2018094316. In one embodiment, stability of a formulation is determined by chromatography, such as size exclusion chromatograph, e.g., size exclusion high performance liquid chromatography (SE-HPLC) or size exclusion ultra high performance liquid chromatography (SE-UHPLC), or hydrophobic high performance liquid chromatography (HI-HPLC), in which a lower change or difference in a first peak from a first formulation before a stress process and/or storage condition as compared to a second peak from the same formulation after the stress process and/or storage condition as compared to a second formulation with a greater change or difference in its first and second peaks before and after a stress process and/or storage condition, respectively, indicates the first formulation is more stable than the second formation.

In another embodiment, stability of a formulation is determined by the turbidity of the formulation (e.g., such as measured at OD₄₀₅ nm), percent of protein recovered (e.g., determined by size exclusion HPLC (SE-HPLC)), and/or purity of protein (e.g., determined by SE-HPLC), in which lower turbidity, higher percentage of recovery and higher purity indicates higher stability. In some embodiments, SDS-PAGE (reducing or non-reducing) is used to determine the stability of a formulation. In some embodiments, asymmetric flow field-flow fractionation (AF4) is used. In other embodiments, isoelectric focusing (IEF), e.g., capiliary isoelectric focusing (cIEF), is used. Increased fragments and/or changes in IEF in a first formulation as compared to a second formulation would indicate the first formulation is less stable. Any one method or combination of methods can be used to determine the stability of a formulation.

The detailed description and following examples illustrate the present invention and are not to be construed as limiting the present invention thereto. Various changes and modifications can be made by those skilled in the art on the basis of the description of the invention, and such changes and modifications are also included in the present invention.

### EXAMPLES

### Example 1: Stability of Formulations With Varving Buffers, Stabilizers, Tonicity Agents and pH

The formulations with 40 mg/ml of aflibercept as shown in Table 1 were prepared and stored at 25° C. for 4 weeks and 40° C. for 2 weeks.

TABLE 1
Formulations A-L.

| Formu- | Aflibercept | | Tonicity | | | |
|---|---|---|---|---|---|---|
| lation | (mg/mL) | Buffer | Agent | Stabilizer | Surfactant | pH |
| A | 40 | 10 mM | | 280 mM | 0.03% PS20 | 6.2 |
| | | Histidine | | Proline | | |
| B | 40 | 10 mM | | 280 mM | 0.03% PS20 | 6.2 |
| | | Potassium | | Proline | | |
| | | phosphate | | | | |
| C | 40 | 10 mM | 40 mM | 200 mM | 0.03% PS20 | 6.2 |
| | | Histidine | NaCl | Proline | | |
| D | 40 | 10 mM | 40 mM | 200 mM | 0.03% PS20 | 6.2 |
| | | Histidine | KCl | Proline | | |
| E | 40 | 20 mM | 140 mM | | 0.005% PS80 | 6.2 |
| | | Sodium | NaCl | | | |
| | | phosphate | | | | |
| F | 40 | 20 mM | | 280 mM | 0.005% PS80 | 6.2 |
| | | Sodium | | Glycine | | |
| | | phosphate | | | | |
| G | 40 | 10 mM | 40 mM | 5% | 0.03% PS20 | 6.2 |
| | | Histidine | NaCl | Sucrose | | |
| H | 40 | 10 mM | | 8% | 0.03% PS20 | 6.2 |
| | | Histidine | | Sucrose | | |
| I | 40 | 10 mM | 40 mM | 200 mM | 0.1% Pluronic | 6.2 |
| | | Histidine | NaCl | Proline | F68 | |
| J | 40 | 10 mM | 40mM | 5% | 0.03% PS20 | 6.2 |
| | | Sodium | NaCl | Sucrose | | |
| | | phosphate | | | | |
| K | 40 | 10 mM | | 10% α,α- | 0.01% PS20 | 5.5 |
| | | Histidine | | trehalose | | |
| | | | | dihydrate | | |
| L | 40 | 10 mM | | 9% | 0.01% PS80 | 5.2 |
| | | acetate | | sucrose | | |

To determine the stability of the formulations described in Table 1, the formulations were tested by Size Exclusion Ultra High Performance Liquid Chromatography (SE-UHPLC). SEC-UHPLC separates proteins based on differences in their hydrodynamic volumes. Molecules with larger hydrodynamic volumes elute earlier than molecules with smaller volumes. The samples were loaded onto an SE-UHPLC column, separated isocratically and the eluent monitored by UV absorbance. Purity was determined by calculating the percentage of each separated component as compared to the total integrated area. The higher the main peak value (e.g., percentage) determined by SEC-UHPLC for a formulation, the more stable the formulation as it indicates a lower level of aggregation. Another indication of increased stability is a lack of change in the main peak value between an initial timepoint and a later timepoint as compared to another formulation.

The percentage of the main peak for each formulation in Table 1 was determined at timepoints 0 and 4 weeks (T=0 and T=4 weeks, respectively) for the formulations stored at 25° C., and timepoints 0 and 2 weeks (T=0 and T=2 weeks, respectively) for the formulations stored at 40° C., as shown in Table 2. The difference or delta value between the main peak percentages of T=0 and T=4 weeks for the formulations stored at 25° C., and at T=0 and T=2 weeks for the formulations stored at 40° C. is also shown in Table 2.

TABLE 2
SEC-UHPLC Main Peak Results for Formulations A-L.

| | | T = 4 week | Delta from | T = 2 week | Delta from |
|---|---|---|---|---|---|
| Formulation | T = 0 | (25° C.) | T = 0 | (40° C.) | T = 0 |
| A | 96.8 | 95.5 | 1.3 | 88.7 | 8.1 |
| B | 96.6 | 95.2 | 1.5 | 86.9 | 9.7 |
| C | 96.8 | 95.7 | 1.1 | 87.0 | 9.8 |
| D | 96.8 | 95.9 | 1.0 | 86.5 | 10.3 |
| E | 96.6 | 95.5 | 1.2 | 85.9 | 10.8 |
| F | 95.9 | 94.5 | 1.5 | 89.0 | 6.9 |
| G | 96.7 | 95.6 | 1.1 | 89.1 | 7.6 |
| H | 96.7 | 95.4 | 1.3 | 88.8 | 8.0 |
| I | 96.8 | 95.9 | 0.9 | 88.7 | 8.1 |
| J | 96.5 | 95.2 | 1.3 | 87.4 | 9.2 |
| K | 97.1 | 96.6 | 0.5 | 94.7 | 2.3 |
| L | 97.1 | 96.6 | 0.5 | 93.8 | 3.2 |

An improved stability profile is demonstrated by a reduction in aggregation levels, thus a higher main peak value and a lower delta value. Surprisingly, formulations with a lower pH (e.g., Formulations K and L) demonstrated a significantly improved stability profile.

### Example 2: Comparison of Formulations With Varying pH

To further test the effect of pH on the stability of the formulations, the same formulation composition but one at a higher pH (e.g., pH 6.4) and one at lower pH (e.g., pH 5.7) was tested. The formulations with 40 mg/ml of aflibercept as shown in Table 3 were prepared and stored at 25° C. for 4 weeks and 40° C. for 2 weeks.

TABLE 3
Formulations 1-10.

| | Aflibercept | | | | |
|---|---|---|---|---|---|
| Formulation | (mg/mL) | Buffer | Stabilizer | Surfactant | pH |
| 1 | 40 | 10 mM Histidine | 280 mM Proline | 0.03% PS20 | 6.4 |
| 2 | 40 | 10 mM Histidine | 280 mM Proline | 0.03% PS20 | 5.7 |
| 3 | 40 | 10 mM Histidine | 8% Sucrose | 0.03% PS20 | 6.4 |
| 4 | 40 | 10 mM Histidine | 8% Sucrose | 0.03% PS20 | 5.7 |
| 5 | 40 | 10 mM Histidine | 4% Sucrose + 4% α,α- | 0.03% PS20 | 6.4 |
| | | | trehalose dihydrate | | |
| 6 | 40 | 10 mM Histidine | 4% Sucrose + 4% α,α- | 0.03% PS20 | 5.8 |
| | | | trehalose dihydrate | | |
| 7 | 40 | 10 mM Histidine | 8% Sucrose | 0.1% Pluronic | 6.4 |
| | | | | F68 | |
| 8 | 40 | 10 mM Histidine | 8% Sucrose | 0.1% Pluronic | 5.7 |
| | | | | F68 | |
| 9 | 40 | 10 mM Acetate | 9% Sucrose | 0.01% PS80 | 5.9 |
| 10 | 40 | 10 mM Acetate | 9% Sucrose | 0.01% PS80 | 5.5 |

To determine the stability of the formulations described in Table 3, the formulations were tested by SEC-UHPLC, as described in Example 1. The higher the main peak value (e.g., percentage) determined by SEC-UHPLC for a formulation, the more stable the formulation as it indicates a lower level of aggregation. Another indication of increased stability is a lack of change in the main peak value between an initial timepoint and a later timepoint as compared to another formulation.

The percentage of the main peak for each formulation in Table 3 was determined at timepoints 0 and 4 weeks (T=0 and T=4 weeks, respectively) for the formulations stored at 25° C., and timepoints 0 and 2 weeks (T=0 and T=2 weeks, respectively) for the formulations stored at 40° C., as shown in Table 4. The difference or delta value between the main peak percentages of T=0 and T=4 weeks for the formulations stored at 25° C., and at T=0 and T=2 weeks for the formulations stored at 40° C. is also shown in Table 4.

TABLE 4
SEC-UHPLC Main Peak Results for Formulations 1-10.

| | | T = 4 week | Delta from | T = 2 week | Delta from |
|---|---|---|---|---|---|
| Formulation | T = 0 | (25° C.) | T = 0 | (40° C.) | T = 0 |
| 1 | 96.3 | 95.3 | 1.0 | 86.1 | 10.2 |
| 2 | 96.6 | 96.5 | 0.1 | 89.5 | 7.1 |
| 3 | 96.2 | 95.1 | 1.1 | 88.4 | 7.8 |
| 4 | 96.6 | 96.4 | 0.2 | 92.7 | 3.9 |
| 5 | 96.1 | 95.1 | 1.0 | 88.7 | 7.4 |
| 6 | 96.5 | 96.2 | 0.3 | 91.3 | 5.2 |
| 7 | 96.2 | 95.5 | 0.7 | 91.0 | 5.2 |
| 8 | 96.6 | 96.4 | 0.2 | 92.7 | 3.9 |
| 9 | 96.4 | 95.5 | 0.9 | 92.1 | 4.3 |
| 10 | 96.7 | 96.5 | 0.2 | 93.6 | 3.1 |

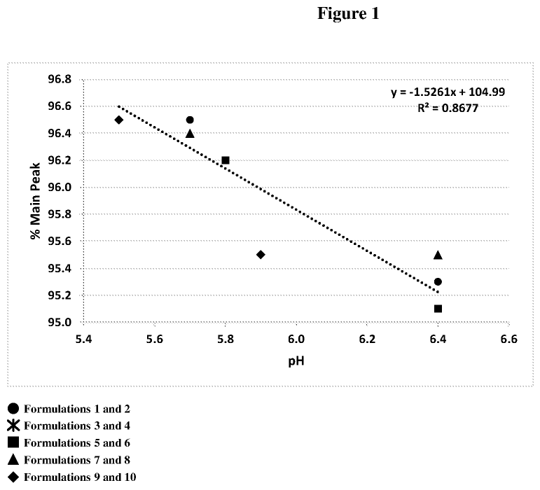

An improved stability profile is demonstrated by a reduction in aggregation levels, thus a higher main peak value and a lower delta value. Formulations with a lower pH demonstrated a significantly improved stability profile, both with a higher main peak value as compared to the same formulation but at a higher pH (see also FIG. 1, which shows the correlation between the pH and the aggregation level of aflibercept at 25° C.), as well as a lower delta value.

### Example 3: Stability of Larger Scale Histidine Buffer Formulations

A study was performed to compare the stability of various formulations at larger scale and the effect of pH under different process stresses as well as long-term storage. The process stresses are used to mimic the manufacturing process. The formulations tested are shown in Table 5. Three groups of formulations were tested: Formulations 11-12 (Formulation 11 is the same as Formulation H); Formulations 13-15, and Formulations 16 and 17. Each group had the same formulation components, with each formulation in the group having a different pH.

TABLE 5
Formulations 11-17.

| | Aflibercept | | | | |
|---|---|---|---|---|---|
| Formulation | (mg/mL) | Buffer | Stabilizer | Surfactant | pH |
| 11 | 40 | 10 mM Histidine | 8% sucrose | 0.03% PS20 | 6.2 |
| 12 | 40 | 10 mM Histidine | 8% sucrose | 0.03% PS20 | 5.5 |
| 13 | 40 | 10 mM Histidine | 4% sucrose + 4% α,α- | 0.01% PS80 | 6.2 |
| | | | trehalose dihydrate | | |
| 14 | 40 | 10 mM Histidine | 4% sucrose + 4% α,α- | 0.01% PS80 | 5.8 |
| | | | trehalose dihydrate | | |
| 15 | 40 | 10 mM Histidine | 4% sucrose + 4% α,α- | 0.01% PS80 | 5.5 |
| | | | trehalose dihydrate | | |
| 16 | 40 | 10 mM Histidine | 4% sucrose + 4% α,α- | 0.1% Pluronic ® | 6.2 |
| | | | trehalose dihydrate | F68 | |
| 17 | 40 | 10 mM Histidine | 4% sucrose + 4% α,α- | 0.1% Pluronic ® | 5.5 |
| | | | trehalose dihydrate | F68 | |

The formulations in Table 5 were prepared at a larger scale than those in Examples 1 and 2 (formulations in Examples 1 and 2 were buffer exchanged with a total volume of about one to two mL for each formulation, whereas formulations at larger scale were buffer exchanged with a total volume of about 200 mL for each formulation). Buffer exchange was performed followed by addition of surfactant. The formulations were then filtrated into Celsius® FFT (Flexible Freeze & Thaw) bags (Sartorius, Germany). Three freeze-thaw cycles were performed. The formulations were then pressure driven filtrated into a stainless steel hold tank and then filtrated by using a peristaltic pump. ISO 2R vials were then manually filled and exposed to one day of light before undergoing transportation simulations. The vials were then placed at 40° C., 25° C., 5° C., and −30° C. for long-term storage. At pre-determined time points, the stability of the samples was tested.

To determine the stability of the formulations described in Table 5, the formulations were tested by SEC-UHPLC, as described in Example 1. The higher the main peak value (e.g., percentage) determined by SEC-UHPLC for a formulation, the more stable the formulation as it indicates a lower level of aggregation. Another indication of increased stability is a lack of change in the main peak value between an initial timepoint and a later timepoint as compared to another formulation.

The percentage of the main peak for each formulation in Table 5 was determined at timepoints 0, 1 week, 2 weeks, and 4 weeks (T=0, T=1 week, T=2 weeks, and T=4 weeks, respectively) for the formulations stored at 40° C., as shown in Table 6. The difference or delta value between the main peak percentages of T=0 and T=1 week, T=2 weeks, or T=4 weeks, respectively, for the formulations stored at 40° C. is also shown in Table 6.

TABLE 6
SEC-UHPLC Main Peak Results for Formulations 11-17.

| | | | | | Delta | Delta | Delta |
|---|---|---|---|---|---|---|---|
| | | | | | Irons | from | from |
| Formu- | | T = 1 | T = 2 | T = 4 | T = 0 | T = 0 | T = 0 |
| lation | T = 0 | weeks | weeks | weeks | (1 week) | (2 weeks) | (4 weeks) |
| 11 | 97.5 | 96.7 | 95.0 | 92.6 | 0.8 | 2.5 | 5.0 |
| 12 | 97.9 | 97.4 | 96.0 | 93.8 | 0.6 | 1.9 | 4.1 |
| 13 | 97.6 | 96.8 | 95.4 | 93.1 | 0.8 | 2.2 | 4.5 |
| 14 | 97.6 | 97.1 | 95.4 | 92.7 | 0.6 | 2.2 | 4.9 |
| 15 | 97.9 | 97.3 | 95.8 | 93.0 | 0.6 | 2.1 | 4.9 |
| 16 | 97.3 | 96.4 | 95.3 | 92.8 | 0.9 | 2.0 | 4.5 |
| 17 | 97.6 | 97.2 | 96.1 | 94.0 | 0.4 | 1.5 | 3.6 |

The formulations with lower pH (Formulations 12, 15, and 17) showed reduced aggregation at T=0 as compared to the higher pH formulations (Formulations 11, 13 and 16, respectively). Furthermore, for two of the three groups of formulations, there was a lower difference in the main peak value between T=0 and T=4 weeks: 4.1 vs 5.0 (Formulation 12 vs Formulation 11) and 3.6 vs 4.5 (Formulation 17 vs Formulation 16). While the third group (Formulations 13-15) did not see a decrease in the difference in the main peak value between T=0 and T=4 weeks, and actually saw an increase, the increase from 4.5 to 4.9 was to a smaller degree (0.4) as compared to the decease (of 0.9) seen for the other two groups.

### Example 4: Stability of Larger Scale Buffer Formulations

A study was performed to compare the stability of various formulations at larger scale and the effect of pH under different process stresses as well as long-term storage. The process stresses are used to mimic the manufacturing process. The formulations tested are shown in Table 7 (Formulation 24 is the same as Formulation K).

TABLE 7
Formulations 18-26.

| | Aflibercept | | | | | |
|---|---|---|---|---|---|---|
| Formulation | (mg/mL) | Buffer | Tonicity | Stabilizer | Surfactant | pH |
| 18 | 40 | 10 mM Sodium | 40 mM | 5% sucrose | 0.03% PS20 | 6.2 |
| | | phosphate | NaCl | | | |
| 19 | 40 | 10 mM Sodium | | 4% sucrose + 4% α,α- | 0.01% PS80 | 6.2 |
| | | phosphate | | trehalose dihydrate | | |
| 20 | 40 | 10 mM Histidine | | 165 mM Arginine | 0.03% PS20 | 6.2 |
| 21 | 40 | 10 mM Histidine | 40 mM | 5% sucrose | 0.03% PS20 | 6.2 |
| | | | NaCl | | | |
| 22 | 40 | 10 mM Histidine | 40 mM | 5% sucrose | 0.01% PS80 | 6.2 |
| | | | NaCl | | | |
| 23 | 40 | 10 mM Histidine | | 9% α,α-trehalose | 0.01%PS80 | 5.8 |
| | | | | dihydrate | | |
| 24 | 40 | 10 mM Histidine | | 10% α,α-trehalose | 0.01% PS20 | 5.5 |
| | | | | dihydrate | | |
| 25 | 40 | 10 mM acetate | | 9% sucrose | 0.01% PS80 | 5.5 |
| 26 | 40 | 10 mM acetate | | 5% sucrose + 4% α,α- | 0.01% PS80 | 5.5 |
| | | | | trehalose dihydrate | | |

The formulations in Table 7 were prepared at a larger scale as in Example 3. Buffer exchange was performed followed by addition of surfactant. The formulations were then filtrated into Celsius® FFT (Flexible Freeze & Thaw) bags (Sartorius, Germany). Three freeze-thaw cycles were performed. The formulations were then pressure driven filtrated into a stainless steel hold tank and then filtrated by a peristaltic pump. ISO 2R vials were then manually filled and exposed to one day of light before undergoing transportation simulations. The vials were then placed at 40° C., 25° C., 5° C., and −30° C. for long-term storage. At pre-determined time points, the stability of the samples was tested.

To determine the stability of the formulations described in Table 7, the formulations were tested by SEC-UHPLC, as described in Example 1. The higher the main peak value (e.g., percentage) determined by SEC-UHPLC for a formulation, the more stable the formulation as it indicates a lower level of aggregation. Another indication of increased stability is a lack of change in the main peak value between an initial timepoint and a later timepoint as compared to another formulation.

The percentage of the main peak for each formulation in Table 7 was determined at timepoints 0, 1 week, 2 weeks, and 4 weeks (T=0, T=1 week, T=2 weeks, and T=4 weeks, respectively) for the formulations stored at 40° C., as shown in Table 6. The difference or delta value between the main peak percentages of T=0 and T=1 week, T=2 weeks, or T=4 weeks, respectively, for the formulations stored at 40° C. is also shown in Table 8.

TABLE 8
SEC-UHPLC Main Peak Results for Formulations 18-26.

| | | | | | Delta | Delta | Delta |
|---|---|---|---|---|---|---|---|
| | | | | | from | from | from |
| Formu- | | T = | T = | T = | T = (1 | T = 0 (2 | T = 0 (4 |
| lation | T = 0 | 1 weeks | 2 weeks | 4 weeks | week) | weeks) | weeks) |
| 18 | 97.3 | 94.9 | 92.1 | 87.5 | 2.3 | 5.2 | 9.8 |
| 19 | 97.2 | 95.8 | 94.5 | 91.2 | 1.4 | 2 7 | 6.0 |
| 20 | 97.6 | 89.5 | 81.5 | 71.3 | 8.1 | 16.1 | 26.3 |
| 21 | 97.3 | 94.7 | 91.8 | 85.4 | 2.6 | 5.5 | 11.9 |
| 22 | 97.5 | 94.9 | 91.9 | 85.6 | 2.6 | 5.6 | 11.8 |
| 23 | 97.6 | 96.9 | 95.4 | 92.7 | 0.6 | 2.2 | 4.9 |
| 24 | 97.9 | 97.4 | 96.3 | 94.3 | 0,5 | 1.6 | 3.6 |
| 25 | 97.9 | 97.5 | 96.4 | 93 8 | 0.5 | 1.6 | 4.1 |
| 26 | 98.0 | 97.4 | 96.0 | 93.7 | 0.6 | 2.0 | 4.3 |

The formulations with the lowest pH (Formulations 23-26, with a pH of 5.5-5.8), had the lowest difference in the main peak value between T=0 and T=4 weeks. Furthermore, the lowest difference was seen in Formulations 24-26, the formulations having the lowest pH tested (5.5).

While the present invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

All references cited in this application are expressly incorporated by reference herein for any purpose.

What is claimed is:

1. A formulation comprising:
a) a fusion protein comprising a domain of a vascular endothelial growth factor (VEGF) receptor and an Fc domain;
b) a buffer, wherein the buffer is a histidine or acetate buffer;
c) a stabilizer; and
d) optionally, a surfactant;
and having a pH between 5.3 and 5.7.

2. The formulation of claim 1, wherein the fusion protein is aflibercept.

3. The formulation of claim 2, wherein the concentration of aflibercept is between 1 and 50 mg/ml mg/ml.

4. The formulation of claim 3, wherein the concentration of aflibercept is about 40 mg/ml.

5. The formulation of claim 4, wherein the buffer is a histidine buffer, and the formulation has a pH between 5.0 and 5.7.

6. The formulation of claim 4, wherein the buffer is an acetate buffer, and the formulation has a pH between 5.3 and 5.7.

7. The formulation of claim 5 or 6, wherein the buffer concentration is about 10 mM.

8. The formulation of claim 5 or 6, wherein the concentration of the stabilizer is between 2% to 10% (w/v).

9. The formulation of claim 8, wherein the stabilizer is an amino acid or sugar.

10. The formulation of claim 9, wherein the stabilizer is proline, glycine, sucrose, sorbitol, glycerol, trehalose, mannitol, dextrose, glucose or any combination thereof.

11. The formulation of claim 10, wherein the stabilizer is sucrose and trehalose.

12. The formulation of claim 11, wherein the formulation comprises a surfactant.

13. The formulation of claim 12, where the surfactant is polysorbate 20, polysorbate 80 or Pluronic F68.

14. The formulation of any one of claims 1-13, further comprising a tonicity agent.

15. The formulation of claim 14, wherein the tonicity agent is sodium chloride or potassium chloride.

16. The formulation of claim 15, wherein the concentration of the tonicity agent is between 40 mM and 140 mM.


SEQUENCE LISTING (2 sequences)

SEQ ID NO: 1 - protein, 431 aa, synthetic construct
  REGION 1..431: note = Fusion protein.
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 432 aa, synthetic construct
  REGION 1..432: note = Fusion protein.
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK